(19) United States
(12) Patent Application Publication
(10) Pub. No.: US 2025/0218575 A1
(43) Pub. Date: Jul. 3, 2025

(54) METHOD AND DEVICE FOR PROVIDING CLINICAL EVALUATION INFORMATION BY USING IMAGE

(71) Applicant: SEOUL NATIONAL UNIVERSITY HOSPITAL, Seoul (KR)

(72) Inventors: Keewon KIM, Gwacheon-si (KR); Sun Gun CHUNG, Seoul (KR); Ri YU, Seoul (KR); Sun Hyung KIM, Seoul (KR)

(73) Assignee: SEOUL NATIONAL UNIVERSITY HOSPITAL, Seoul (KR)

(21) Appl. No.: 19/101,779

(22) PCT Filed: Dec. 23, 2022
(86) PCT No.: PCT/KR2022/021146

§ 371 Date: Feb. 6, 2025

(30) Foreign Application Priority Data
Dec. 24, 2021 (KR) 10-2021-0186664

(51) Int. Cl. G16H 30/40; G06T 7/246; G06T 7/70; G16H 50/30

(52) U.S. Cl. CPC G16H 30/40; G06T 7/251; G06T 7/70; G16H 50/30

(57) ABSTRACT

A method and device for providing clinical evaluation information by using an image are provided. The method for providing clinical evaluation information by using an image includes the steps of: actively or passively acquiring a photographed image, estimating a 3D posture of a person using only one of depth information and 2D body coordinate information of the person, obtained based on the photographed image, or by integrating the two pieces of information, analyzing a clinical determination criteria indicator according to the person's posture, on the basis of the 3D posture to analyze the person's motion, and generating and providing person's clinical evaluation information based on the clinical determination criteria indicator.

### TECHNICAL FIELD

The present disclosure relates to a method and device for providing clinical evaluation information using images.

### BACKGROUND ART

As society transitions into an aging population, the demand for healthcare services for elderly individuals living at home has significantly increased. In particular, when the decline in physical performance due to aging makes independent daily living difficult, elderly individuals may no longer be able to reside at home, leading to reliance on nursing homes or significantly increasing the caregiving burden on their families. The physical performance refers to functional parameters objectively measured in relation to bodily movements, such as walking speed, balance ability, and muscle strength. The decline in physical performance is directly affected by sarcopenia and is closely linked to the progression of frailty. The worsening of frailty often leads to a vicious cycle of chronic disease exacerbation, resulting in substantial medical and social costs. Among the most common factors directly leading to functional decline and frailty in older adults is falling, which occurs due to a complex interplay of factors such as motor function, cognitive ability, and balance. Falling is the leading cause of trauma requiring hospitalization and is a major contributor to mortality in older adults. The evaluation of fall risk, along with the prediction and prevention of falls, is essential for promoting healthy aging in older individuals. If evaluations of physical performance, fall risk, and screening for frailty or sarcopenia were conducted earlier and at more appropriate times, it could lead to reduced medical costs for elderly individuals living at home and improvements in healthcare systems centered on service providers.

Quantitative analysis of human movements can directly extract physical performance parameters and evaluate fall risk. Additionally, physical performance can be directly applied to sarcopenia screening tests, and various parameters can be integrated to screen and monitor the onset and progression of frailty. In clinical settings, short physical performance battery (SPPB) tests are commonly performed to evaluate physical performance, while the Berg Balance Scale is used to evaluate balance ability. For fall risk evaluation, gait stability can also be evaluated. However, this often requires expensive motion analysis equipment, making it difficult to utilize widely. Diagnostic tests for sarcopenia typically include evaluations such as walking speed and chair-stand tests. Physical performance parameters are also critically considered in frailty evaluations. All of these tests are conducted in clinical environments by medical professionals, and to ensure objective and quantitative reliability, qualitative evaluations of movement are excluded, focusing only on simple metrics such as time and distance. For example, only the time taken to stand up and sit down five times is evaluated, without considering differences in pose. Since these evaluations rely on visual observations by medical professionals and are conducted in clinical settings, additional effort and time are required for appointment scheduling, result recording, reporting, and review. Moreover, these evaluations are typically conducted as one-time measurements, resulting in low correlation with physical performance in real-life settings and making continuous evaluation difficult. As a result, it is currently practically impossible to conduct most of these tests at an early stage for screening or prevention purposes before significant problems arise.

### DETAILED DESCRIPTION OF THE INVENTION

### Techinical Problem

The objective of the present disclosure is to recognize and analyze the movement of a subject person without a user's effort or awareness using a camera installed in a home, thereby enabling a non-intrusive, non-invasive, and continuous assessment of physical function and fall risk and a frailty/sarcopenia screening test.

The objective of the present disclosure is to provide a method and a device for providing clinical evaluation information, which prevent falls in advance and detect frailty and sarcopenia early by deriving clinical evaluation information (physical function indicator, fall risk, frailty and sarcopenia) from an image.

In addition, the objective of the present disclosure is to provide a method and a device for providing clinical evaluation information, which shorten an examination time and enable more precise and accurate analysis by automatically deriving clinical evaluation information from a motion image using artificial intelligence technology.

However, problems to be solved by the present disclosure are may not be limited to the above-described problems. Although not described herein, other problems to be solved by the present disclosure can be clearly understood by those skilled in the art from the following description.

### Technical Solution

According to an aspect of the present disclosure to solve the above-described problem, a method for providing clinical evaluation information by using an image, the method being performed by a device includes acquiring a captured image representing a person from at least one image sensor, estimating a three-dimensional pose of the person by acquiring at least one of RGB image information and depth information for each of body parts of the person, based on the captured image and acquiring three-dimensional (3D) body coordinate information based on the at least one information, analyzing a motion of the person by analyzing clinical judgment criteria indicators according to a pose of the person based on the three-dimensional pose, and generating and providing clinical evaluation information including at least one of the person's physical function score, sarcopenia status, fall risk, and frailty status based on the clinical judgment criteria indicators.

According to an aspect of the present disclosure, a device for providing clinical evaluation information by using an image includes an image acquisition unit that acquires a captured image representing a person from at least one image sensor, a pose estimation unit that estimates a three-dimensional pose of the person by acquiring at least one of RGB image information and depth information for each of body parts of the person, based on the captured image and acquire three-dimensional (3D) body coordinate information based on the at least one information, a motion analysis unit that analyzes a motion of the person by analyzing clinical judgment criteria indicators according to the pose of the person based on the three- dimensional pose, and a clinical evaluation information provision unit that generates and provides clinical evaluation information including at least one of the person's physical function score, sarcopenia status, fall risk, and frailty status based on the clinical judgment criteria indicators.

Other specific details of the inventive concept are included in the detailed description and drawings.

### Advantageous Effects of the Invention

As mentioned above, it is possible to prevent falls in advance and ensure safety by deriving clinical evaluation information (physical function indicator, fall risk, frailty and sarcopenia) from images.

Further, as mentioned above, since clinical evaluation information is automatically derived from motion images using artificial intelligence technology, it is possible to shorten the examination time and enable more precise and accurate analysis by providing clinical evaluation information more quickly and accurately.

However, effects of the present disclosure may not be limited to the above-described effects. Although not described herein, other effects of the present disclosure can be clearly understood by those skilled in the art from the following description.

However, effects of the present disclosure may not be limited to the above-described effects. Although not described herein, other effects of the present disclosure can be clearly understood by those skilled in the art from the following description.

### DESCRIPTION OF THE DRAWINGS

FIG. 1 is a diagram for describing a clinical evaluation information provision system according to an embodiment of the present disclosure.

FIG. 2 is a block diagram for describing a device for providing clinical evaluation information according to an embodiment of the present disclosure.

FIG. 3 is a block diagram for describing an example of a motion classification method and a clinical judgment criteria indicators method according to an embodiment of the present disclosure.

FIG. 4 illustrates a flowchart for describing a method for providing clinical evaluation information according to an embodiment of the present disclosure.

FIG. 5 is a flow chart for describing one embodiment of step of acquiring the captured image and step of estimating a three-dimensional pose of a person, which are shown in FIG. 4.

FIG. 6 is a flow chart for describing another embodiment of step of acquiring a captured image and step of estimating a three-dimensional pose of a person, which are shown in FIG. 4.

FIG. 7 is a graph illustrating an embodiment of a clinical judgment criteria indicator.

FIG. 8 is a flow chart for describing a modification embodiment of the method of providing clinical evaluation information shown in FIG. 4.

### BEST MODE

Advantages and features of the present disclosure and methods for achieving them will be apparent with reference to embodiments described below in detail in conjunction with the accompanying drawings. However, the present disclosure is not limited to the embodiments disclosed below, but may be implemented in various forms, and these embodiments are to make the disclosure of the present disclosure complete, and are provided so that this disclosure will be thorough and complete and will fully convey the scope of the invention to those of ordinary skill in the art, which is to be defined only by the scope of the claims.

The terminology used herein is for the purpose of describing particular embodiments only and is not intended to be limiting of the present disclosure. The singular expressions include plural expressions unless the context clearly dictates otherwise. In this specification, the terms “comprises” and/or “comprising” are intended to specify the presence of stated elements, but do not preclude the presence or addition of elements. Like reference numerals refer to like elements throughout the specification, and “and/or” includes each and all combinations of one or more of the mentioned elements. Although “first”, “second”, and the like are used to describe various components, these components are of course not limited by these terms. These terms are only used to distinguish one component from another. Thus, a first element discussed below could be termed a second element without departing from the teachings of the present disclosure.

Unless otherwise defined, all terms (including technical and scientific terms) used herein have the same meaning as commonly understood by one of ordinary skill in the art to which this invention belongs. It will be further understood that terms such as those defined in commonly used dictionaries, will not be interpreted in an idealized or overly formal sense unless expressly so defined herein.

Spatially relative terms, such as “below”, “beneath”, “lower”, “above”, and “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” may encompass both an orientation of above and below. Components may also be oriented in other orientations, and thus spatially relative terms may be interpreted according to orientations.

Hereinafter, embodiments of the present disclosure will be described in detail with reference to the accompanying drawings.

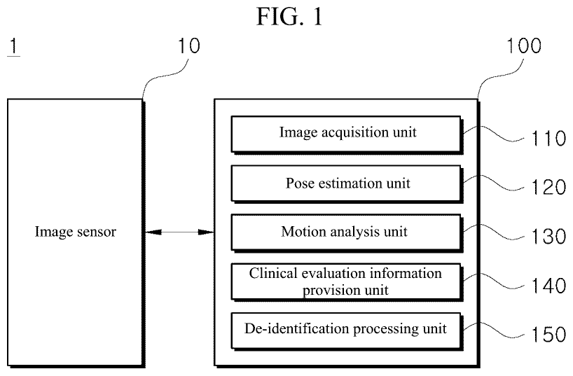

FIG. 1 is a diagram for describing a clinical evaluation information provision system 1 according to an embodiment of the present disclosure.

Referring to FIG. 1, the clinical evaluation information provision system 1 may provide clinical information about a subject using images obtained by photographing the subject. The subject may be, for example, a human, but is not limited thereto. In the following, it is assumed that the subject is a human.

The clinical evaluation information provision system 1 may collect movements performed by a person in daily life with an image sensor (e.g., an installed camera), and then monitor information on physical performance and fall risk at all times without detention.

The clinical evaluation information provision system 1 may collect images of movements performed by a person in daily life without any particular purpose, recognize a specific person, replace the specific person with de-identified information, recognize the person through a pose estimation algorithm, classify and extract a specific movement, reconstruct and quantify the movement, and provide clinical evaluation information for use in human health management.

Specifically, the clinical evaluation information provision system 1 may identify an individual by recognizing a person subject to evaluation, detect a movement useful for evaluation, extract a quantitative parameter of physical performance and fall risk from the movement, and propose personalized health care to the person based on the parameters of physical performance for screening for sarcopenia and monitoring for frailty.

The clinical evaluation information provision system 1 may be applied to healthcare- related biomedical engineering technologies, and may be applied in the fields of remote healthcare and smart healthcare to monitor a user's physical performance and implement appropriate interventions based on the physical performance. However, the present disclosure is not limited thereto.

The clinical evaluation information provision system 1 may include an image sensor 10 and a clinical evaluation information provision device 100.

The image sensor 10 may capture a person, an object, an environment, or the like. The image sensor 10 may generate a captured image obtained by capturing the person, the object, the environment and the like. The image sensor 10 may provide the captured image to the clinical evaluation information provision device 100.

For example, the image sensor 10 may be a camera that includes a depth sensor or an infrared irradiation depth camera.

The depth sensor may sense depth information of an object.

In another example, the image sensor 10 may be a LiDAR (Light Detection And Ranging). In this case, the LiDAR may sense depth information like a depth sensor.

In another example, the image sensor 10 may be an RGB camera that provides RGB image information.

The image sensor 10 may be embedded in a mobile device owned by a person being captured.

Alternatively, the image sensor 10 may be embedded in an imaging device (e.g., smartphone, smartpad, security camera, depth camera, etc.) owned by a measurer other than a person being captured. The captured image may include at least one of a still image (or a photograph, snapshot image, static image, etc.) and a moving image. For example, the captured image may be a moving image. However, the present disclosure is not limited thereto.

The clinical evaluation information provision device 100 may receive the captured image from the image sensor 10 and provide clinical evaluation information about the person being captured. The clinical evaluation information may include, for example, but is not limited to, the person's physical performance score, sarcopenia status, fall risk, and frailty status.

The clinical evaluation information provision device 100 may include an image acquisition unit 110, a pose estimation unit 120, a motion analysis unit 130, a clinical evaluation information provision unit 140, and a de-identification processing unit 150.

The image acquisition unit 110 may acquire a captured image representative of a person from the image sensor 10. In an embodiment, the captured image may be an image of a person performing predefined poses, taken either to provide clinical evaluation information or to generate a clinical judgment reference metric.

For such captured images, the captured image may be generated in such a way that a person subject to be captured adopts the aforementioned poses and captures himself or herself using the image sensor 10 he or she possess.

Alternatively, the captured image may be generated by capturing the person that adopts the aforementioned poses with the image sensor 10 owned by an external organization, such as a hospital.

In other embodiments, the captured image may be an image that is captured by the image sensor 10 in real time. For such captured image, the captured image may be generated by capturing a person present in a capturing area in real time with the image sensor 10 owned by an external organization, such as a hospital. As used herein, a ‘pose’ may be stationary or in motion.

The aforementioned poses may refer to states representing stillness or movement, such as a walking pose (gait), a stand-up pose, a standing pose, a turning-around pose or a hand- reaching pose.

The pose as described herein may also be referred to as an action, motion, behaviour, or the like that indicates stillness or movement. In addition, the captured image may be obtained by automatically classifying, using an algorithm, a portion of the images captured in real time, in which the person takes a specified motion/pose while the user is performing daily life motions without being conscious of the evaluation.

In an embodiment, when the image sensor 10 is an RGB camera, the pose estimation unit 120 may obtain RGB image information from the captured image. The pose estimation unit 120 may obtain three-dimensional body coordinate information from the RGB image information using a three-dimensional body coordinate extraction algorithm.

The three-dimensional coordinate extraction algorithm may include, for example, but is not limited to, an algorithm using a sagittal plane, a frontal plane, a transverse plane, or an algorithm using human anatomical information.

In other embodiments, when the image sensor 10 is a depth sensor or LiDAR, the pose estimation unit 120 may obtain depth information from the captured image. The pose estimation unit 120 may obtain three-dimensional body coordinate information based on the depth information.

In another embodiment, the pose estimation unit 120 may obtain the three-dimensional body coordinate information using only RGB image information, by capturing anatomical information about a human skeleton, or by an algorithm that estimates the volume/contours of a three-dimensional object.

In another embodiment, the pose estimation unit 120 may obtain two-dimensional body coordinate information of a person and depth information for each of the body parts of the person based on the captured image.

The pose estimation unit 120 may integrate (or fuse) the two-dimensional body coordinate information and the depth information to estimate a three-dimensional pose of the person.

In an embodiment, the pose estimation unit 120 may obtain a plurality of two-dimensional body coordinates from the captured image as two-dimensional body coordinate information using a two-dimensional pose estimation algorithm. Here, the two-dimensional pose estimation algorithm may be, but is not limited to, OpenPose or PoseNet.

The motion analysis unit 130 may analyze a human motion by analyzing clinical judgment criteria indicators based on the human pose based on the three-dimensional pose. The clinical judgment criteria indicators may include gait speed, gait steadiness, cadence variability, stride variability, standing-up speed, trunk inclination, quadriceps strength, upper limb support, balance retention time, center of mass sway and sagittal plane balance, center of mass excursion and change of gait steadiness, center of mass sway, center of mass excursion, and range of motion of upper extremity.

However, the present disclosure is not limited thereto. The clinical judgment criteria indicators may be determined based on the pose of the person shown in the captured image.

For example, in a gait pose, the clinical judgment criteria indicators may include gait speed, gait steadiness, cadence variability, and stride variability.

In another example, in a stand-up pose, the clinical judgment criteria indicators may include standing-up speed, trunk inclination, quadriceps strength, and upper limb support. In another example, in a standing pose, the clinical judgment criteria indicators may include center of mass sway and sagittal plane balance.

In another example, in a turning pose, the clinical judgment criteria indicators may include center of mass excursion and change of gait steadiness. In another example, in a hand- reaching pose, the clinical judgment criteria indicators may include balance retention time, center of mass sway, center of mass excursion, range of motion of the upper extremity.

The clinical evaluation information provision unit 140 may generate and provide clinical evaluation information based on the clinical judgment criteria indicators. Here, the clinical evaluation information may include, but is not limited to, at least one of a person's gait metrics, balance, physical function score, sarcopenia status, fall risk, and frailty status. The clinical evaluation information may be used for healthcare purposes.

The de-identification processing unit 150 may de-identify, based on artificial intelligence, body parts of a person, which are usable to identify the person, in a captured image, such that the person to be captured is de-identified.

Specifically, for example, the de-identification processing unit 150 may apply an AI-based blurring technique or masking technique to a person's eyes or other parts that the person is sensitive to, such as a face or body scars, to avoid violating privacy laws. This has the effect of protecting the privacy of the person being captured.

A computer program performing the method of providing clinical evaluation information of the clinical evaluation information provision system 1 may be stored on a non-transitory computer-readable recording medium.

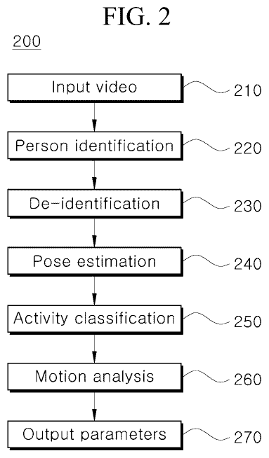

FIG. 2 is a block diagram for describing a device for providing clinical evaluation information according to an embodiment of the present disclosure.

Referring to FIGS. 1 and 2, a device for providing clinical evaluation information 200 may receive a video (input video 210). The video may be a captured image as described above. By way of specific example with reference to FIGS. 1 and 2, the image acquisition unit 110 may receive a video (e.g., captured images) from the image sensor 10 (see FIG. 1).

The device for providing clinical evaluation information 200 may identify a person included in the captured image (Person Identification, 220). Here, identifying a person included in the captured image (220) may be detecting a subject person among an object, a surrounding, and a person included in the captured image.

Further, identifying a person may include determining whether the person in the captured image is one of the users previously evaluated or one of the pre-entered subject persons (e.g., family members, hospital patients).

By way of specific example, with reference to FIGS. 1 and 2, the image acquisition unit 110 may identify the person by detecting the person being captured in the captured image. Further, identifying the person may include determining whether the person included in the captured image is one of users previously evaluated or one of a pre-entered (or preset) subject persons (e.g., family members).

The device for providing clinical evaluation information 200 may de-identify the person identified in the captured image (de-identification, 230). By way of specific example with reference to FIGS. 1 and 2, the de-identification processing unit 150 may blur certain body parts of a person (e.g., eyes and the area around the eyes) in the captured image.

The device for providing clinical evaluation information 200 may estimate the pose of the de-identified person and classify the activity (or pose) being performed (Pose estimation, 240 and Activity classification, 250). As a specific example with reference to FIGS. 1 and 2, the pose estimation unit 120 may estimate a three-dimensional pose of the person based on the captured image, and the motion analysis unit 130 may classify the person's motion based on the estimated three-dimensional pose using artificial intelligence.

The device for providing clinical evaluation information 200 may analyze the classified motion and output clinical judgment criteria indicators according to the classified motion (Motion analysis, 260 and Output Parameters, 270). As a specific example with reference to FIGS. 1 and 2, the motion analysis unit 130 may extract clinical judgment criteria indicators according to the classified motion.

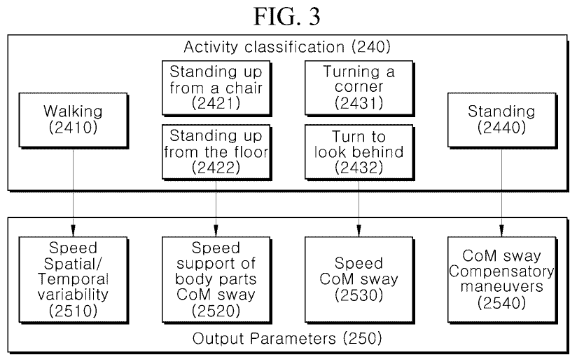

FIG. 3 is a block diagram for describing an example of a motion classification method and a clinical judgment criteria indicators method according to an embodiment of the present disclosure.

Referring to FIGS. 2 and 3, motions (or poses) as described herein may include walking (2410), standing up from a chair (2421), standing up from the floor (2422), turning a corner (2431), turning to look behind (2432), and standing (2440).

However, the present disclosure is not limited thereto. The motions (or poses) may further include standing up from the floor without touching the floor, spinning in place, changing direction while walking, picking up objects from the floor, hand-reaching out, and the like.

Herein, standing (2440) may include balancing on both feet, balancing on one foot, and tandem standing and the like. Such motions (or poses) may reflect certain distances or maintenance times as conditions.

In this specification, walking (2410) is referred to as a walking pose; standing up from a chair (2421) and standing up from the floor (2422) are referred to as a stand-up pose; turning a corner (2431) and turning to look behind (2432) are referred to as a turning-around pose; and standing (2440) is referred to as a standing pose.

The standing pose may include, for example, a pose of standing on one foot or a pose of standing on two feet. The pose of standing on two feet may include poses where the feet are freely placed on the ground for foot spacing and placement, such as both feet aligned in a straight line parallel to the walking direction or aligned perpendicularly to the walking direction.

When the motion of the person in the captured image is Walking (2410), the motion analysis unit 130 may extract first clinical evaluation parameters (2510), which include gait speed, gait steadiness, center of mass (COM; also referred to as the center of gravity), COM motion, and gait pattern. Meanwhile, the gait steadiness may include spatial variability and temporal variability.

When the motion of the person in the captured image is Standing up from a chair (2421) or Standing up from the floor (2422), the motion analysis unit 130 may extract second clinical evaluation parameters 2520, which include a time required for motion change (e.g., standing-up speed), the number of repetitions, trunk inclination, support of body parts, and CoM sway.

When the motion of the person in the captured image is Turning a corner (2431) or Turning to look behind (2432), the motion analysis unit 130 may extract third clinical evaluation parameters 2530, which include COM sway/excursion and change of gait steadiness.

When the motion of the person in the captured image is Standing (2440), the motion analysis unit 130 may extract fourth clinical evaluation parameters 2540, which include COM sway and sagittal alignment/balance.

The foregoing embodiments are merely examples and are not limited to FIG. 3. The following embodiments and other implementations will be described later.

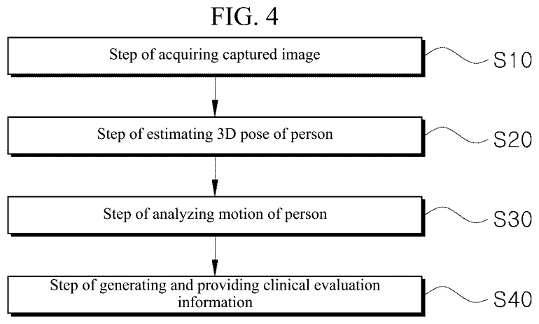

FIG. 4 illustrates a flowchart for describing a method for providing clinical evaluation information according to an embodiment of the present disclosure.

Referring to FIG. 4, the method for providing clinical evaluation information may include acquiring a captured image (S10), estimating a three-dimensional (3D) pose of a person (S20), analyzing the person's motion (S30), and generating and providing clinical evaluation information (S40).

Step S10 may be performed by the image acquisition unit 110 described with reference to FIG. 1.

Step S20 may be performed by the pose estimation unit 120 described with reference to FIG. 1.

Step S30 may be performed by the motion analysis unit 130 described with reference to FIG. 1.

In one embodiment, a person's pose may include at least one of a walking pose, a stand-up pose, a standing pose (including a pose of balancing while standing), a turning-around pose, or a hand-reaching pose. The analyzing of the motion of a person (S30) may analyze, in the case of a walking pose, gait speed, gait steadiness, and gait pattern as clinical judgment criteria indicators. The gait steadiness may include temporal variability (e.g., cadence variability) and spatial variability (e.g., stride length variability or step length variability). The gait patterns may include a limping gait, a steppage gait, a hemiplegic gait, a parkinsonian gait, a waddling gait, and a crouch gait.

Alternatively, the analyzing of the motion of a person (S30) may analyze, in the case of a standing-up pose, a time required for motion change (e.g., the speed of standing up and sitting down), the number of repetitions, quadriceps strength, trunk inclination, or upper limb support, and CoM sway as clinical judgment criteria indicators.

Alternatively, the analyzing of the motion of a person (S30) may analyze, in the case of a standing pose, center of mass sway and sagittal plane balance, as clinical judgment criteria indicators.

Alternatively, the analyzing of the motion of a person (S30) may analyze, in the case of turning-around pose, center of mass excursion and change of gait variability/steadiness, as clinical judgment criteria indicators. Alternatively, the analyzing of the motion of a person (S30) may analyze, in the case of hand-reaching pose, range of motion of the upper extremity, center of mass sway, and center of mass excursion, as clinical judgment criteria indicators.

Step S40 may be a step performed by the clinical evaluation information provision unit 140 described above with reference to FIG. 1. In an embodiment, the generating and providing of the clinical evaluation information (S40) may include measuring a physical function score and determining sarcopenia status based on gait speed, and deriving a fall risk based on gait steadiness.

In an embodiment, the generating and providing of the clinical evaluation information (S40) may include measuring a physical function score based on a standing-up speed, and calculating a lower body strength of the person based on trunk inclination, quadriceps strength, and upper limb support to determine whether the person has sarcopenia.

In an embodiment, the generating and providing of the clinical evaluation information (S40) may evaluate balance ability or fall risk, and measure progression of degenerative spine disease based on center of mass sway and sagittal plane balance.

In an embodiment, the generating and providing of the clinical evaluation information (S40) may measure joint range of motion and evaluate joint disease.

In another embodiment, the generating and providing of the clinical evaluation information (S40) may include calculating the correlation between clinical evaluation information derived by designers, experimenters, or physicians and clinical evaluation information derived using artificial intelligence to generate calibrated clinical evaluation information. Accordingly, more accurate analyses are possible, and thus more accurate information is delivered.

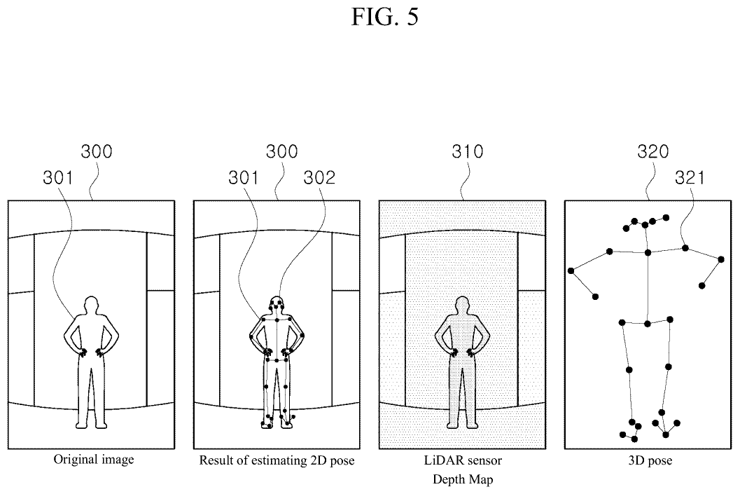

FIG. 5 is a flow chart for describing one embodiment of step of acquiring a captured image and step of estimating a three-dimensional pose of a person, which are shown in FIG. 4.

Referring to FIG. 5, the captured image may be an image 300 representing a state in which a person 301 has performed a pre-directed motion to extract a clinical judgment criteria indicator.

The pre-directed motion to extract the clinical judgment criteria indicator may be a motion that is instructed to the person 301 by a measurer other than the person 301, for example. The image 300 may be an image taken directly by the person 301, or may be an image taken of the person 301 by a measurer other than the person 301 (or an imaging device owned by the measurer).

For example, referring to FIG. 5, in an original image, the person 301 included in the image 300 may take a standing pose. In this case, the image acquisition unit 110 may acquire the image 300 as a captured image.

The pose estimation unit 120 may perform a two-dimensional pose estimation algorithm using the image 300 as input. As a result of the two-dimensional pose estimation, the pose estimation unit 120 may obtain a plurality of two-dimensional body coordinates from the image 300 as two-dimensional body coordinate information 302.

The plurality of two-dimensional body coordinates included in the two-dimensional body coordinate information 302 may be coordinates located at a head, chest, shoulder, arm, torso, leg, or the like of the person 301. The number of the plurality of two-dimensional body coordinates may be, for example, 25, but is not limited thereto.

The pose estimation unit 120 may obtain depth information 310 from the image 300. Referring to FIGS. 1 and 5, for example, when the image sensor 10 includes a LiDAR sensor, the depth information 310 may be obtained by receiving a depth map processed by the LiDAR sensor.

The pose estimation unit 120 may integrate the two-dimensional body coordinate information 302 and the depth information 310 to generate three-dimensional coordinate information 321 including three-dimensional coordinates. The pose estimation unit 120 may estimate the three-dimensional pose of the person 301 using the three-dimensional coordinate information 321.

The pose estimation unit 120 may generate the three-dimensional body coordinate information 321 based on an algorithm, directly from the two-dimensional person image 301, or directly from the body coordinate information 302.

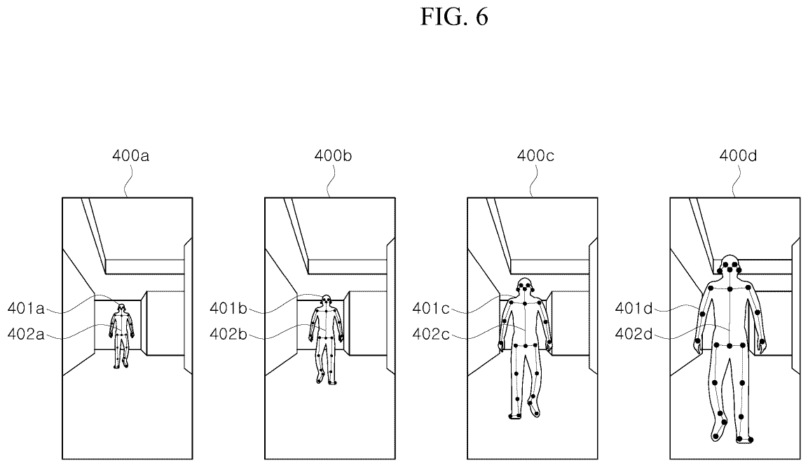

FIG. 6 is a flow chart for describing another embodiment of step of acquiring a captured image and step of estimating a three-dimensional pose of a person, which are shown in FIG. 4.

Referring to FIG. 6, the captured image may be a real-time image 400a, 400b, 400c, or 400d of a person 401a, 401b, 401c, or 401d captured by the image sensor 10 in real time. The real-time image 400a, 400b, 400c, or 400d may be an image taken of the person 401a, 401b, 401c, or 401d by a measurement device.

It is assumed that the drawings illustrated in FIG. 6 represent a sequential flow of time. In this case, it is assumed that the person 401a, 401b, 401c, or 401d is taking a walking pose, progressing from the leftmost diagram to the rightmost diagram shown in FIG. 6.

The image acquisition unit 110 may acquire an image (e.g., the image 300 shown in FIG. 5) representing the pre-dictated motion from the real-time image 400a, 400b, 400c, or 400d as a captured image.

The pose estimation unit 120 may acquire two-dimensional body coordinate information 402a, 402b, 402c, or 402d of the person 401a, 401b, 401c, or 401d based on the captured image acquired from the real-time image 400a, 400b, 400c, or 400d, similarly as described above with reference to FIG. 5.

Also similarly as described above with reference to FIG. 5, the pose estimation unit 120 may obtain depth information based on the captured image acquired from the real-time image 400a, 400b, 400c, or 400d, and estimate a three-dimensional pose of the person 401a, 401b, 401c, or 401d by integrating the depth information with the two-dimensional body coordinate information 402a, 402b, 402c, or 402d.

Alternatively, the pose estimation unit 120 may estimate a three-dimensional pose based on an algorithm, directly from the two-dimensional person image 301 or directly from the body coordinate information 302.

The embodiment illustrated in FIG. 6 may be referred to as ambient monitoring of physical function. The ambient monitoring of physical function refers to a methodology that enables the continuous evaluation of physical function and fall risk, as well as the ongoing tracking of frailty and sarcopenia, through the analysis of daily life activities by analyzing motions in daily life in a non-invasive and non-restrictive manner based on knowledge of clinical body function assessment.

Accordingly, it is possible to recognize and analyze daily life activities in a non-invasive and non-restrictive manner to enable clinically significant physical function assessments, thus allowing easy use without requiring special knowledge or intent from the user to user-friendly and effectively provide continuous monitoring of the user's physical function.

In addition, it is possible to provide clinically necessary information easily and efficiently compared to evaluation in a conventional hospital environment or evaluation using a wearable device.

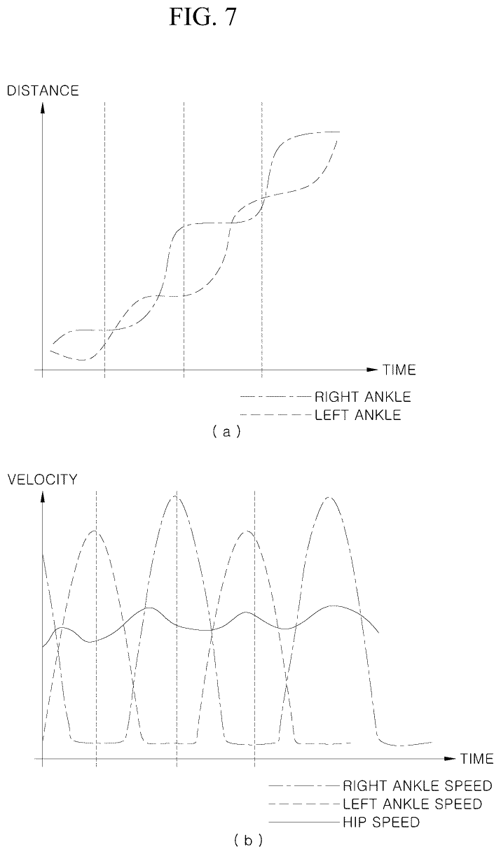

FIG. 7 is a graph illustrating an embodiment of a clinical judgment criteria indicator.

Referring to FIG. 7, the clinical judgment criteria indicator may be determined based on the pose of a person in a captured image.

For example, in a gait pose, the clinical judgment criteria indicators may include gait speed, gait steadiness, stride variability, and cadence variability. In another example, in a stand-up pose, the clinical judgment criteria indicators may include standing-up speed, trunk inclination, quadriceps strength, and upper limb support. In another example, in a standing pose, the clinical judgment criteria indicators may include balance retention time, center of mass sway and sagittal plane balance. In another example, in a turning pose, the clinical judgment criteria indicators may include center of mass excursion and change of gait steadiness. In another example, in a hand-reaching pose, the clinical judgment criteria indicators may include center of mass sway, center of mass excursion, and the range of motion of the upper extremity.

Referring to (a) of FIG. 7, for example, when the pose of a person is a walking pose, the motion analysis unit 130 may obtain three-dimensional coordinates of each of the person's right ankle RIGHT ANKL and left ankle LEFT ANKLE. The motion analysis unit 130 may measure the distance of the three-dimensional coordinates of each of the right ankle and left ankle over time. The distance of the three-dimensional coordinates of both ankles, a right ankle and a left ankle over time may be represented as the graph shown in (a) of FIG. 7.

Referring to (b) of FIG. 7, for example, when the pose of a person is a walking pose, the motion analysis unit 130 may measure a right ankle speed, a left ankle speed, and a hip speed of the person based on the three-dimensional coordinates.

However, without limitation, the motion analysis unit 130 may measure joint-specific kinematic parameters as needed, such as left knee speed, right knee speed, right ankle displacement, and left hip angular velocity.

The right ankle kinematic parameter, left ankle kinematic parameter, and a hip kinematic parameter over time may be expressed as the graph shown in (b) of FIG. 7.

Here, the kinematic parameters may be, for example, displacement (or distance traveled) per joint, linear velocity (or linear acceleration) per joint, or angular velocity per joint, and may include linear velocity and angular velocity per joint, and angular displacement per joint. However, the kinematic parameters may include, but are not limited to, a variety of kinematic geometric variables.

Depending on an embodiment, the motion analysis unit 130 may also measure torsion, torque, and the like.

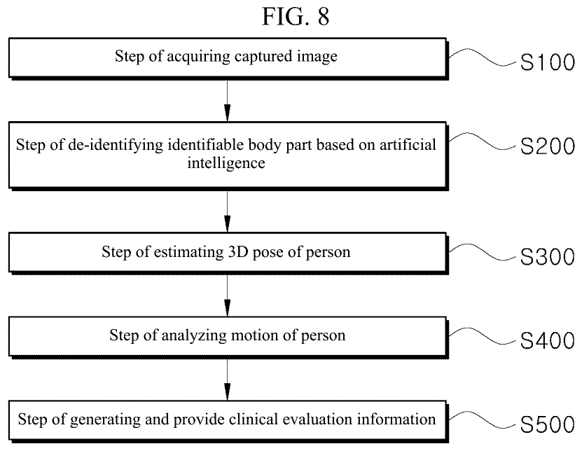

FIG. 8 is a flow chart for describing a modification embodiment of the method of providing clinical evaluation information shown in FIG. 4.

Referring to FIG. 8, the method of providing clinical evaluation information shown in FIG. 8 may include acquiring a captured image (S100), de-identifying an identifiable body part based on artificial intelligence (S200), estimating a three-dimensional pose of a person (S300), analyzing a motion of the person (S400), and generating and providing clinical evaluation information (S500). Step S100, step S300, step S400, and step S500 illustrated in FIG. 8 correspond to step S10, step S20, step S30, and step S40 illustrated in FIG. 4, respectively, and will not be described herein.

Step S200 may be a step performed by the de-identification processing unit 150 described above with reference to FIG. 1. Specifically, after the operation S100 of acquiring a captured image, the de-identifying of the body part usable to identify the person among body parts in the captured image is performed based on artificial intelligence.

As described above, by deriving clinical evaluation information (physical function parameters, fall risk, senility, and sarcopenia) from the captured image, it is possible to prevent falls in advance, thereby promoting safety.

Furthermore, as described above, since the clinical evaluation information is automatically derived from motion images using artificial intelligence technology, the clinical evaluation information is provided faster and more accurately, thereby shortening the examination time and enabling more precise and accurate analysis.

According to the present disclosure, it is possible to objectively present clinically useful parameters in real time by simply capturing a specific motion with a mobile phone or a portable device (tablet PC, Kinect, etc.).

In addition, it is possible to quantitatively evaluate the characteristics of a motion through three-dimensional motion analysis, thereby providing a quantitative parameter for significant clinical information such as fall risk. In addition, a mobile application has been developed, which may be used as a tool for users to easily diagnose sarcopenia or identify frailty.

In addition, anyone with a smartphone or tablet PC may take images and access the images through the mobile application of the present disclosure, thereby reducing the cost of testing. The present disclosure also provides convenience to people by allowing patients and the elderly to evaluate their physical capabilities at home without having to visit a doctor.

In addition, the present disclosure provides convenience to people by being used for telemedicine for elderly patients, patients with limited mobility, and patients in mountainous areas who are unable to visit due to COVID-19 (Coronavirus).

In addition, the present disclosure has the effect of promoting health by continuously monitoring one's physical health state by periodically using an evaluation tool applied to the present disclosure. The present disclosure provides convenience to clinicians by enabling clinicians to evaluate the physical capabilities of patients by replacing existing clinical evaluation tools in cases of suspected frailty or sarcopenia.

In addition, the present disclosure enables healthcare providers to reduce the human and time resources expended in assessing physical function, and users to more easily and regularly monitor their own physical function. The present disclosure also allows for earlier identification of individuals with physical decline who may require intervention.

The steps of a method or algorithm described in connection with the embodiments of the present disclosure may be implemented directly in hardware, in a software module executed by hardware, or in a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or in a computer readable recording medium that is well known in the art.

Although embodiments of the present disclosure have been described above with reference to the accompanying drawings, it is understood that those skilled in the art to which the present disclosure pertains may implement the present disclosure in other specific forms without changing the technical spirit or essential features thereof. Therefore, it should be understood that the embodiments described above are illustrative in all respects and not restrictive.

1. A method for providing clinical evaluation information by using an image, the method being performed by a device, comprising:
acquiring a captured image representing a person from at least one image sensor; and
estimating a three-dimensional pose of the person by acquiring at least one of RGB image information and depth information for each of body parts of the person, based on the captured image and acquiring three-dimensional (3D) body coordinate information based on the at least one information;
analyzing a motion of the person by analyzing clinical judgment criteria indicators according to a pose of the person based on the three-dimensional pose; and
generating and providing clinical evaluation information including at least one of the person's physical function score, sarcopenia status, fall risk, and frailty status based on the clinical judgment criteria indicators.

2. The method of claim 1, wherein the estimating of the three-dimensional pose of the person includes
acquiring the RGB image information and the depth information based on the captured image, acquiring a plurality of two-dimensional body coordinates from the RGB image information as two-dimensional body coordinate information using a two-dimensional pose estimation algorithm, and acquiring the three-dimensional body coordinate information by integrating the two-dimensional body coordinate information and the depth information; or
capturing anatomical information about human skeleton, or acquiring the three-dimensional body coordinate information through an algorithm for estimating volume/contours of a three-dimensional object, by using the RGB image information alone.

3. The method of claim 2, wherein the pose of the person includes at least one of a walking pose, a stand-up pose, a standing pose, a turning-around pose, or a hand-reaching pose, and
wherein the analyzing of the motion of the person includes
analyzing a gait speed, gait steadiness, Center of Mass (COM) motion and gait pattern in the walking pose,
analyzing standing-up speed, trunk inclination, support of body parts, and CoM sway in the stand-up pose,
analyzing balance retention time, CoM sway and sagittal plane balance in the standing pose,
analyzing COM sway and change of gait steadiness in the turning-around pose, and
analyzing COM sway and range of motion of upper extremity in the hand-reaching pose.

4. The method of claim 3, wherein the generating and providing of the clinical evaluation information includes
measuring the physical function score and determining the sarcopenia status based on the gait speed, and deriving the fall risk based on the gait steadiness;
measuring the physical function score based on the standing-up speed, and calculating a lower body strength of the person based on the trunk inclination, and the support of the body part to determine the sarcopenia status; and
measuring the physical function score based on the COM sway and the sagittal plane balance.

5. The method of claim 4, wherein the captured image is an image representing a state in which the person has performed a pre-directed motion to extract the clinical judgment criteria indicator,
wherein the acquiring of the captured image includes acquiring the image as the captured image, and
wherein the estimating of the three-dimensional pose of the person includes acquiring the two-dimensional body coordinate information and the depth information based on the image.

6. The method of claim 4, wherein the captured image is a real-time image of the person captured by the at least one image sensor in real time,
wherein the acquiring of the captured image includes
acquiring, as the captured image, an image representing a pre-directed motion to extract the clinical judgment criteria indicator, from the real-time image, or
acquiring, from an image captured in real time, a portion of the image in which the person takes a specified motion/pose while a user is performing daily life motions without being conscious of the evaluation by using an algorithm.

7. The method of claim 1, further comprising:
after the acquiring of the captured image, de-identifying a body part usable to identify the person among body parts in the captured image based on artificial intelligence.

8. The method of claim 1, wherein the at least one image sensor includes an RGB camera, a depth sensor, and a LiDAR.

9. The method of claim 1, wherein the estimating of the three-dimensional pose of the person includes acquiring the RGB image information from the captured image and acquiring the three-dimensional body coordinate information from the RGB image information using an algorithm for extracting three-dimensional coordinates.

10. The method of claim 1, wherein the estimating of the three-dimensional pose of the person includes acquiring the depth information from the captured image and acquiring the three-dimensional body coordinate information based on the depth information.

11. A device for providing clinical evaluation information by using an image, comprising:
an image acquisition unit configured to acquire a captured image representing a person from at least one image sensor;
a pose estimation unit configured to estimate a three-dimensional pose of the person by acquiring at least one of RGB image information and depth information for each of body parts of the person, based on the captured image and acquiring three-dimensional (3D) body coordinate information based on the at least one information;
a motion analysis unit configured to analyze a motion of the person by analyzing clinical judgment criteria indicators according to the pose of the person based on the three-dimensional pose; and
a clinical evaluation information provision unit configured to generate and provide the clinical evaluation information including at least one of the person's physical function score, sarcopenia status, fall risk, and frailty status based on the clinical judgment criteria indicators.

12. The device of claim 11, wherein the pose estimation unit is configured to, when estimating the 3D pose of the person,
acquire the RGB image information and the depth information based on the captured image, acquire a plurality of two-dimensional body coordinates from the RGB image information as two-dimensional body coordinate information using a two-dimensional pose estimation algorithm, and acquire the three-dimensional body coordinate information by integrating the two-dimensional body coordinate information and the depth information; or acquire 3D body coordinate information by using the RGB image information based on anatomical characteristics of the person or estimation of contours/volume of an object, by using the RGB image information alone.

13. The device of claim 12, wherein the pose of the person includes at least one of a walking pose, a stand-up pose, a standing pose, a turning-around pose, or a hand-reaching pose, and
wherein the motion analysis unit is configured to, when analyzing the motion of the person,
analyze a gait speed, gait steadiness, Center of Mass (COM) motion and gait pattern in the walking pose,
analyze standing-up speed, trunk inclination, support of body parts, and CoM sway in the stand-up pose,
analyze COM sway and sagittal plane balance in the standing pose,
analyze COM sway and change of gait steadiness in the turning-around pose, and
analyze COM sway and range of motion of upper extremity in the hand-reaching pose.

14. The device of claim 13, wherein the clinical evaluation information provision unit is configured to, when generating and providing the clinical evaluation information,
measure the physical function score and determine the sarcopenia status based on the gait speed, and derive the fall risk based on the gait steadiness;
measure the physical function score based on the standing-up speed, and calculate a lower body strength of the person based on the trunk inclination, and the support of body part to determine the sarcopenia status; and
measure the physical function score based on the balance retention time, the COM sway and the sagittal plane balance.

15. The device of claim 11, further comprising:
after the acquisition of the captured image, a de-identification unit configured to de-identify a body part usable to identify the person among body parts in the captured image based on artificial intelligence.
